(19) United States
(12) Patent Application Publication
(10) Pub. No.: US 2020/0205900 A1
(43) Pub. Date: Jul. 2, 2020

(54) DYNAMIC 3D MOTION CAPTURE FOR SURGICAL IMPLANT ORIENTATION

(71) Applicant: Motion Insights, Inc., Allendale, NJ (US)

(72) Inventors: Aaron J. Buckland, New York, NY (US); Sang J. Kim, Allendale, NJ (US); Themistocles S. Protopsaltis, Englewood Cliffs, NJ (US); Jonathan M. Vigdorchik, Brooklyn, NY (US)

(21) Appl. No.: 16/730,526

(22) Filed: Dec. 30, 2019

Related U.S. Application Data
(60) Provisional application No. 62/786,787, filed on Dec. 31, 2018.

(51) Int. Cl. A61B 34/10; G06T 13/40; G06K 9/00

(52) U.S. Cl. CPC A61B 34/10; G06T 13/40; A61B 2562/0219; A61B 2034/105; A61B 2034/102; G06K 9/00342

(57) ABSTRACT

A processing device receives first continuous motion capture data representing dynamic motion of at least one of a bone or a joint of the subject user while performing a physical movement. The processing device further generates, based on the continuous motion capture data, a three dimensional (3D) visualization comprising a virtual avatar performing the physical movement and provides a visualization of a surgical implant overlaid on the virtual avatar. The processing device receives input comprising an adjustment to an orientation of the visualization of the surgical implant relative to the at least one of the bone or the joint and determines whether the orientation of the visualization of the surgical implant satisfies a condition pertaining to the subject user performing the physical movement.

### RELATED APPLICATIONS

This application claims the benefit of U.S. Provisional Patent Application No. 62/786,787, filed Dec. 31, 2018, the entire contents of which are hereby incorporated by reference herein.

### TECHNICAL FIELD

The present disclosure is generally related to computer systems, and is more specifically related to dynamic 3D motion capture for surgical implant orientation.

### BACKGROUND

Two dimensional (2D) imaging is widely used by doctors and other health professionals to analyze human motion in sports and health applications because 2D imaging is relatively simple, inexpensive and widely available. Three dimensional (3D) motion visualization is much more advanced and provides data, multiple viewing angles, and digital data analysis that 2D imaging cannot provide. 3D systems can provide useful information of angles, speed, orientation, etc. which can be used to identify poor movement for performance or health. 3D motion visualization, however, requires sensors or markers and technology that takes longer to set up and is more expensive.

### BRIEF DESCRIPTION OF THE DRAWINGS

The present disclosure is illustrated by way of example, and not by way of limitation, and can be more fully understood with reference to the following detailed description when considered in connection with the figures in which:

FIG. 1 depicts a high-level component diagram of an illustrative system architecture, in accordance with one or more aspects of the present disclosure.

FIG. 2 is a flow diagram illustrating method of preoperative implant orientation planning in accordance with one or more aspects of the present disclosure.

FIG. 3 is a flow diagram illustrating method of intraoperative implant orientation in accordance with one or more aspects of the present disclosure.

FIG. 4 illustrates one example of a 3D visualization showing a virtual avatar performing a physical movement in accordance with one or more aspects of the present disclosure.

FIG. 5 illustrates another example of a 3D visualization showing a virtual avatar performing a physical movement in accordance with one or more aspects of the present disclosure.

FIG. 6 depicts an example computer system which can perform any one or more of the methods described herein, in accordance with one or more aspects of the present disclosure.

### DETAILED DESCRIPTION

Embodiments for dynamic 3D motion capture for surgical implant orientation. In one embodiment, a framework for optimizing the orientation of a surgical implant using dynamic three-dimensional (3D) motion data captured before, during, and/or after a surgical procedure is described. The 3D motion data can be applied to a proprietary orientation algorithm to determine an orientation configuration for a surgical implant that is personalized to a given patient and potentially optimized for the patient's unique/innate motion characteristics, as well as potentially for their occupation or recreational activities.

Today the vast majority of surgical implant procedures do not involve a preoperative implant orientation plan developed in advance of the surgical procedure. Of those limited number of procedures that do include a preoperative implant orientation plan, the involved technology is rudimentary. At best, a number of two-dimensional (2D) radiographs (i.e., x-ray images) or a computerized tomography scan are captured of the patient's bones or joints in few static positions (e.g., supine, standing, and/or sitting). These static images are less than ideal for planning the orientation of a surgical implant due to their imprecise nature, their ability to capture only a brief moment in time which can be quite variable, and their limited reproducibility and consistency possibly leading to measurement error. The x-ray images generally only capture a single plane (e.g., the sagittal plane, the coronal plane) and do not offer a 3D perspective. In addition, the static images cannot be used to detect rotational (e.g., axial or transverse plane) or coupled orientation changes, and do not offer the ability to discern postoperative kinematic differences relative to the preoperative state of the patient.

The implementations described herein address the above and other considerations by providing for dynamic 3D motion capture for use in surgical implant orientation. In one implementation, a network of one or more 3D motion capture sensors is used to perform 3D motion capture at one or more of three stages, including before, during, and/or after a surgical implant procedure. The surgical implant procedure may include an operation to provide a patient with a surgical implant, such as a hip replacement, knee replacement, spinal (e.g., lumbar, thoracic, cervical) fusion, spinal decompression or spinal disc replacement, shoulder replacement, elbow, wrist, ankle replacement, small joint replacement in the hands and feet, or other musculoskeletal procedures. Optimal orientation of the surgical implant with respect to the rest of the patient's body is critical to ensuring a successful surgical outcome and durability. Knowing the patient's actual movement patterns and maximum ranges of motion across multiple planes and movements may reduce the risks of negative side effects including dislocation or implant wear, implant or bony impingement, implant loosening, squeaking and fracturing, metallosis, etc.

In one implementation, the 3D motion capture sensors are used to develop a personalized preoperative implant orientation plan. The 3D motion capture sensors are placed at specific locations on the patient's body relative to where the implant will be located. The sensors can be, for example, wireless sensors including a combination of circuitry, such as accelerometers, gyroscopes, magnetometer, etc., used to perform dynamic 3D motion capture. In one implementation, the 3D motion capture sensors can capture the continuous movement data as the patient takes a number of positions and performs a number of dynamic movements. For example, the 3D motion capture sensors can measure the position of the patient's bones and joints in a supine position, standing position, sitting position, lateral bending, forward or backward bending, flexed seated position, etc. In addition, the motion capture can include continuous measurements taken while the user is transitioning from sitting to standing (getting up from sitting), supine to standing, stepping up, walking, running, tying shoes, etc. Certain occupational or recreational movements can also be measured, such as lifting a box, swinging a golf club, performing yoga poses, riding a bike, etc. Depending on the implementation, the sensors can measure the position of bones including the cervical spine, thoracic spine, lumbar spine, scapula, humerus, radius/ulna, small bones of hands and feet, pelvis, femur, tibia/fibula, talus, etc., and joints including the shoulder, elbow, wrist, atlanto-occipital, cervical spine, thoracic spine, lumbar spine, sacrum, hip, knee, ankle, etc., while these movements are performed. In one implementation to measure movement of the spine, for example, the 3D motion capture sensors can be placed at the occiput, occipito-cervical junction, cervical-thoracic junction, lumbar spine, and sacrum in order to capture motion of the spine including bending side to side (i.e., right/left) and flexion/extension (i.e., forward/backward) of cervical, thoracic, and/or lumbar spine. The 3D motion capture sensors thus provide the freedom to capture more types of movement based on lifestyle and risk activities to allow the system to create a more custom and personalized plan and fit of surgical implants.

Accordingly, the system can perform a method of generating patient 3D inertial motion capture of various patient-specific postures (e.g., supine, standing, sitting, getting up from sitting, walking, etc.). The system can further capture the complex interplay of axial and appendicular skeletal joint motions that exist between them. With any joint motion, there are at least two bones that move with six degrees of freedom, both rotational and translational. Traditional radiographs can only capture these in two dimensions at static points in time. There are, however, multiple other motions and forces that can occur. For example, when a person moves from standing to sitting, there are multiple things that happen including the lumbar spine moving, the pelvis rotating and translating, the proximal femur rotating around the hip center, etc. Every person does these movements differently and in different increments for each of the involved bones and joints. These motions go beyond just a static pose, and can include for example, walking, getting up from sitting, rolling over in bed, twisting when standing, and the like. In addition, the system can measure more complex functional tasks based on the patient's occupation or recreational activities (e.g., a golf swing, playing tennis, stepping on a ladder, performing carpentry on the floor, kneeling, crawling, etc.).

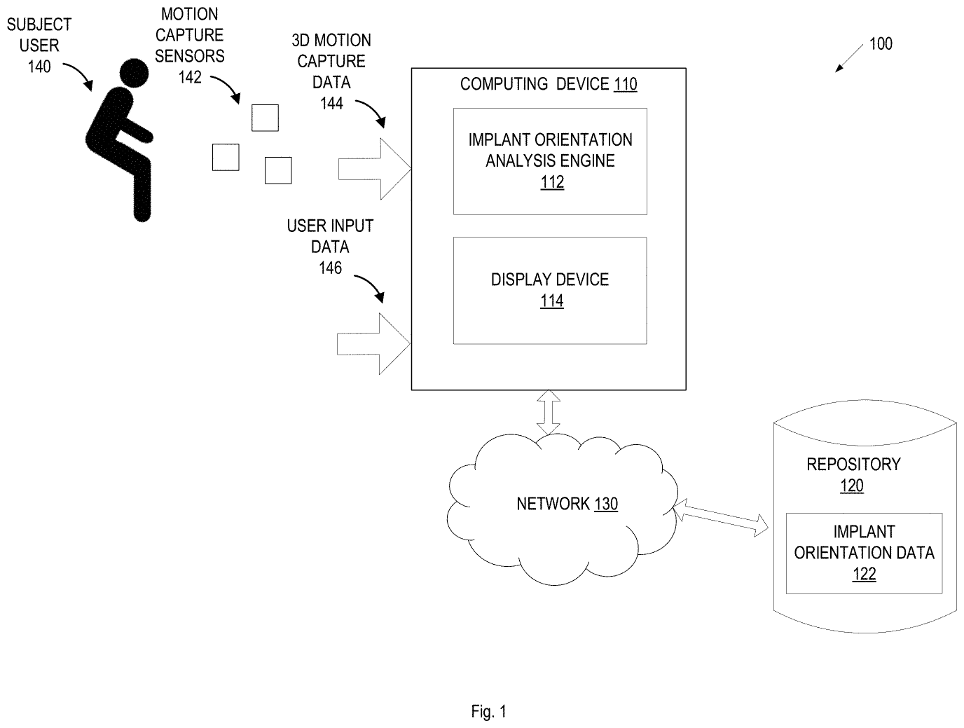

FIG. 1 depicts a high-level component diagram of an illustrative system architecture 100, in accordance with one or more aspects of the present disclosure. System architecture 100 includes a computing device 110 and a repository 120 connected to a network 130. Network 130 may be a public network (e.g., the Internet), a private network (e.g., a local area network (LAN) or wide area network (WAN)), or a combination thereof.

The computing device 110 may be configured to perform dynamic 3D motion capture for surgical implant orientation and/or other analyses. In one embodiment, computing device 110 may be a desktop computer, a laptop computer, a smartphone, a tablet computer, a server, or any suitable computing device capable of performing the techniques described herein. In one embodiment, a plurality of motion capture sensors 142, which may be affixed to one or more body parts of a subject user 140 while they are performing a physical movement, capture 3D motion capture data 144 corresponding to the subject user 140. Depending on the implementation, the motion capture sensors 142 can be attached externally to the skin of the subject user 140 or internally to the bones of the subject user 140. In other embodiments, the motion capture sensors 142 may be affixed to any relevant object being manipulated by the subject user 140 while performing the physical movement, such as to a golf club, baseball bat, tennis racquet, crutches, prosthetics, etc. The 3D motion capture data 144 may be received by the computing device 110.

The 3D motion capture data 144 may be received in any suitable manner. For example, the motion capture sensors 142 may be wireless inertial sensors, each including for example, a gyroscope, magnetometer, accelerometer, and/or other components to measure sensor data including relative positional data, rotational data, and acceleration data. The 3D motion capture data 144 may include this sensor data and/or other data derived or calculated from the sensor data. The motion capture sensors 142 may transmit the 3D motion capture data 144 including, raw sensor data, filtered sensor data, or calculated sensor data, wirelessly to computing device 110 using internal radios or other communication mechanisms. In other embodiments, other systems may be used to capture 3D motion capture data 144, such as an optical system, using one or more cameras, a mechanical motion system, an electro-magnetic system, an infra-red system, etc. In addition, in other embodiments, the 3D motion capture data 144 may have been previously captured and stored in a database or other data store. In this embodiment, computing device 110 may receive the 3D motion capture data 144 from another computing device or storage device where the 3D motion capture data 144 is maintained. In still other embodiments, the 3D motion capture data 144 may be associated with other users besides or in addition to subject user 140 performing a physical activity.

In one embodiment, motion capture sensors 142 capture the 3D motion capture data 144 while the subject user 140 is performing a physical activity or a physical movement. The physical activity can be for example, swinging a golf club, throwing a ball, running, walking, jumping, sitting, standing, or any other physical activity. When performing the physical activity, the subject user 140 may make one or more physical body movements that together enable performance of the physical activity. For example, when swinging a golf club, the user may rotate their hips and shoulders, swing their arms, hinge their wrists, etc., each of which can be considered a separate body movement associated with performing the physical activity. Each physical activity may have its own unique set of associated body movements. Each physical movement can involve motion of a bone or joint of the subject user 140. Thus, the 3D motion capture data 144 can include continuous motion capture data representing dynamic motion of at least one of a bone or joint of the subject user 140 while they are performing the physical movement. The continuous nature can differentiate the 3D motion capture data 144 from a mere static image captured at a single point in time.

In one embodiment, computing device 110 may include an implant orientation analysis engine 112. The implant orientation analysis engine 112 may include instructions stored on one or more tangible, machine-readable storage media of the computing device 110 and executable by one or more processing devices of the computing device 110. In one embodiment, implant orientation analysis engine 112 receives the 3D motion capture data 144 of the subject user 140 performing the physical activity or physical movement and generates a 3D visualization comprising a virtual avatar performing the physical movement, where the 3D visualization is based on the received 3D motion capture data 144. Implant orientation analysis engine 112 further provides a visualization of a surgical implant overlaid on the virtual avatar. In one embodiment, the virtual avatar can be an exact 3D model of the patient's bony or soft tissue anatomy generated based on data imported from radiographs, a CT scan, MRI, 3D ultrasound, or some other source. Collectively, this data can be referred to as skeletal references which can be taken from a known posture which may then be registered to the 3D motion capture data 144. In other embodiments, the virtual avatar can be an approximation or other representation of the patient's anatomy, which may or may not be exact, and which may be based only partially on imported data. The surgical implant can be, for example, a replacement joint including one or more separate components (e.g., a hip joint, knee joint, shoulder joint, intervertebral disc), a prosthesis (e.g. for osteotomy, arthrodesis, fracture fixation, or arthroplasty), or any other type of surgical implant designed to be inserted or affixed to the subject user's body during a surgical procedure. Implant orientation analysis engine 112 causes display (e.g., on display device 114 of computing device 110 on a display device of some other device connected to network 130, or wirelessly connected directly to computing device 110 or any other computing device) of the 3D visualization comprising the virtual avatar performing the physical movement along with the visualization of the surgical implant.

Implant orientation analysis engine 112 may further receive input, such as user input data 146, from a surgeon, or other health professional, including an orientation of the visualization of the surgical implant relative to at least one of a bone or a joint of the virtual avatar. In one embodiment, the visualization of the surgical implant is initially provided at a default orientation which can be adjusted by the received input. In another embodiment, the user input data 146 can specify an initial orientation and geometry for the surgical implant, including a separate orientation for each of the individual components of the surgical implant. For example, for a hip replacement, the user input data 146 can include at least one of an angle of inclination or an angle of anteversion corresponding to at least one component of the surgical implant (acetabular of femoral components). The angle of inclination or an angle of anteversion can be defined according to at least one of a global reference frame, functional pelvic plane, or an anterior pelvic plane (APP) reference frame. In other embodiments, the user input data 146 can include other information, such as a size of the surgical implant, position of the surgical implant, varying geometries of the surgical implant such as neck angle, an offset of the surgical implant, head lengths, or other inputs.

In one embodiment, implant orientation analysis engine 112 determines whether the orientation of the visualization of the surgical implant, either the initial orientation or as adjusted in response to the user input, satisfies a condition pertaining to the subject user performing the physical movement. An output of implant orientation analysis engine 112, such as an optimal orientation, or a determination of whether a given orientation satisfies the condition, can be stored in repository 120 as implant orientation data 122. The condition can include, for example, whether the visualization of the surgical implant will come within a threshold margin of an angle of impingement while the virtual avatar is performing the physical movement. Other conditions that can be evaluated include whether a given orientation improves or maximizes the range of motion, or the contact area between the surgical implant and the bone in order to reduce edge loading and implant wear, whether a given orientation is optimized for stress distribution to prevent wear, loosening, and/or breakage of the surgical implant, whether the center of pressure of a ball component within a cup component of the surgical implant moves too close to the tolerated range of motion in order to prevent dislocation, etc. Additional details regarding the operation of implant orientation analysis engine 112 are provided below.

In other embodiments, the details described above are modified according to the specific procedure to be performed. For the spine, for example, 3D motion capture data 144 can represent an assessment of spinal alignment of the subject user 140 for a variety of postures encountered in daily living. Implant orientation analysis engine 112 can then perform an analysis as part of preoperative planning for spinal deformity correction or spinal disc replacement surgery to predict the stresses applied to the proximal and distal junctions with surgical implants of known materials and density. This analysis can minimize the chances of adjacent segment disease, proximal and distal-junctional kyphosis, proximal and distal junctional failure, and can optimize quality of life outcomes for the subject user 140. Implant orientation analysis engine 112 can further assess dynamic alignment in different activities of daily living and also spine flexibility as in assessing cervical, thoracic and lumbar spine stiffness and flexibility in multiple planes: sagittal, coronal and axial.

In one embodiment, the 3D motion capture data 144, user input data 146, thresholds and other data associated with the condition, and the resulting orientation data that is determined to satisfy the condition can be stored as part of implant orientation data 122 in repository 120. The repository 120 is a persistent storage that is capable of storing implant orientation data 122, as well as data structures to tag, organize, and index this data. Repository 120 may be hosted by one or more storage devices, such as main memory, magnetic or optical storage based disks, tapes or hard drives, NAS, SAN, and so forth. Although depicted as separate from the computing device 110, in an implementation, the repository 120 may be part of the computing device 110 or may be directly attached to computing device 110. In some implementations, repository 120 may be a network-attached file server, while in other embodiments, repository 120 may be some other type of persistent storage such as an object-oriented database, a relational database, and so forth, that may be hosted by a server machine or one or more different machines coupled to the via the network 130.

In one embodiment, implant orientation analysis engine 112 may use a set of trained machine learning models that are trained and used to analyze the 3D motion capture data 144 and provide a recommendation for the subject user 140 pertaining to an optimal orientation of the surgical implant. The implant orientation analysis engine 112 may also preprocess any received 3D motion capture data, such as 3D motion capture data 144, prior to using the data for training of the set of machine learning models and/or applying the set of trained machine learning models to the data. In some instances, the set of trained machine learning models may be part of the implant orientation analysis engine or may be accessed on another machine (e.g., a separate server machine) by the implant orientation analysis engine 112. Based on the output of the set of trained machine learning models, the implant orientation analysis engine 112 may obtain a recommendation for the subject user 140 pertaining to optimal orientation for the surgical implant, such as an orientation that minimizes the likelihood of impingement.

The set of machine learning models may refer to model artifacts that are created by a training engine using the training data that includes training inputs and corresponding target outputs (i.e., correct answers for respective training inputs). During training, patterns in the training data that map the training input to the target output (i.e., the answer to be predicted) can be found, and are subsequently used by the machine learning models for future predictions. The set of machine learning models may be composed of, e.g., a single level of linear or non-linear operations (e.g., a support vector machine [SVM]) or may be a deep network, i.e., a machine learning model that is composed of multiple levels of non-linear operations). Examples of deep networks are neural networks including convolutional neural networks, recurrent neural networks with one or more hidden layers, and fully connected neural networks. Convolutional neural networks include architectures that may provide efficient physical movement analysis. Convolutional neural networks may include several convolutional layers and subsampling layers that apply filters to portions of the data to detect certain attributes/features. Whereas many machine learning models used for personalized recommendations often suffer from a lack of information about users and their behavior, as well as a lack of relevant input data, implant orientation analysis engine 112 has the benefit of high quality information about the users, their physical and demographic attributes, goals and a large amount of movement data. As such, the set of machine learning models, and/or other artificial intelligence models may include, for example, content personalization, collaborative filtering, neural networks or statistical analysis to create high quality implant orientation recommendations to achieve the desired results. This level of information can allow implant orientation analysis engine 112 to make very specific goal based recommendations directed to, for example, more power, speed, accuracy, flexibility, etc.

As noted above, the set of machine learning models may be trained to determine a recommendation for the subject user 140 pertaining to the orientation of a surgical implant. Once the set of machine learning models are trained, the set of machine learning models can be provided to implant orientation analysis engine 112 for analysis of new 3D motion capture data. For example, implant orientation analysis engine 112 may input the motion capture data into the set of machine learning models. The implant orientation analysis engine 112 may then obtain one or more outputs from the set of trained machine learning models.

An example of the acquisition of hip motion capture using 3D motion capture sensors 142 is described below. In one implementation, the sensors capture continuous motion data as the subject user 140 starts supine and moves to standing (e.g., getting out of bed), moves from standing to a sitting position. The sensors can further capture data as the subject user 140 is getting up from sitting to a standing position, and finally walking around the room. Data from all of these movements can be provided to computing device 110 which may analyze the movement data to determine the unique movement patterns of this particular patient and identify an optimal orientation of a surgical implant for the patient. In addition, the server can take other action to further refine the analysis including mating the supine acquisition data to a supine radiograph, mating the supine acquisition data to a CT scan, mating the standing acquisition data to a standing radiograph, mating the acquisition data to an intraoperative radiograph/fluoroscopy/C-arm/O-arm/Ultrasound/CT, or preoperative or postoperative CT, or mating the inertial sensor data 144 to an image which meshes the data to the bony anatomy and provides a reference to the functional position of the bone, which then causes implication for implant orientation. Based on the reference position, and the delta motions between positions, implant orientation analysis engine 112 can determine a functional position of the bones (i.e. femur, pelvis, lumbar spine, etc.) to allow for optimized implant orientation. In one embodiment, such as for a hip replacement procedure, the implant orientation can be defined by acetabular cup anteversion and inclination, femoral torsion/anteversion, functional acetabular anteversion and inclination, and functional femoral torsion. In another embodiment, such as a knee replacement, the 3D motion capture data 144 can represent ligament tensions and up to six degrees of freedom motion between the femur and tibia for example. Implant orientation analysis engine 112 can then extrapolate the orientation for knee replacement components. In this embodiment, the implant orientation can be defined by varus/valgus, flexion/extension, tibial slope, internal/external rotation of the femoral and tibial components. For shoulders, implant orientation analysis engine 112 can consider humeral varus/valgus/torsion, tuberosity height, glenoid orientation for standard or reverse shoulder arthroplasty, etc. For the spine, implant orientation analysis engine 112 can calculate global alignment (SVA/TPA/T1SPi/GSA/Coronal & Axial) versus regional alignment (LL, PT, TK, coronal and axial) changes with posture in order to optimize spinal alignment for the subject user. Other procedures and other implants, can define the orientation differently. From these acquisitions and the functional positions of the bone, implant orientation analysis engine 112 can determine the functional position of the prosthetic implants (e.g., femoral and acetabular components for a hip replacement, femoral and tibial components for a knee replacement, etc. . . . ) throughout the entire range of functional movements, as well as perform an impingement analysis or ligament tension assessment or other analyses, as described in more detail below.

Consider an example situation where the subject user 140 receives a standing lateral X-ray which includes the lumbar spine, pelvis, and femoral shaft. From this X-ray, the anterior pelvic plane (and/or spinopelvic tilt, pelvic incidence, pelvic incidence-lumbar lordosis, combined sagittal angle, etc. . . . ) can be calculated. The anterior pelvic plane here becomes the reference value for 0 degree of anteversion of an acetabular component, and the horizontal and/or transischial line becomes the reference plane for the inclination angle. A theoretical acetabular component position of 40 degrees inclination and 20 degrees of anteversion is placed on the 3D model with reference to the above planes. As the subject user 140 goes through various ranges of motion, that acetabular component will move based on the mobility of the spine and pelvis. Implant orientation analysis engine 112 can calculate exactly how it moves from the 3D motion capture as the subject user 140 goes through various ranges of motion, and based on those motions, can graph the functional positions of the acetabular and femoral components to ensure that within each range of motion, the position of the components does not show any evidence of impingement (or limits the likelihood of impingement occurring) and is within safe zones. However, if the motions do show evidence of impingement, user input data 146 can manually change the orientations or computing device 110 with implant orientation analysis engine 112 can automatically modify the positions to optimize an implant orientation that minimizes impingement.

Once the best fit acetabular component position/orientation is calculated, this will output a value of inclination and anteversion in reference to any available plane (i.e., the anterior pelvic plane, spinopelvic tilt, or any plane like parallel to the floor, parallel to the CT scan bed, parallel to the body, etc.). Anteversion is a number in reference to something (20 degrees, but 20 degrees from 0 degrees) and most navigation systems in use now use the anterior pelvic plane, the global plane of the body, or the plane of the flatbed of the CT scan. So the system can output this value (e.g., 40 inclination, 20 anteversion) in reference to any plane.

In addition, in one implementation, dynamic 3D motion capture can be used intraoperatively (i.e., during a surgical procedure). As described above, 3D motion capture data 144 can be acquired prior to surgery using inertial sensors placed on the subject user 140. In one embodiment, the 3D motion capture data 144 is captured some time prior to the surgical procedure (e.g., days, weeks, or months prior) so that a surgical plan can be developed. In another embodiment, the 3D motion capture data 144 is captured immediately prior to the surgery, such as the same day (e.g., in the operating room or holding area prior to surgery). In addition, the sensors can remain on the subject user 140 during the surgical procedure. By acquiring the position of bones or joints in certain poses prior to surgery, mating them to the pre-operative or intra-operative imaging as described above (e.g., intraoperative registration), and keeping the sensors on during surgery, implant orientation analysis engine 112 can know the position of the body in real-time. Registration is a processing involving mating bone and imaging. In robotic surgery, this can include directly the robot where the bone is and matching that location to a preoperative CT scan, for example. As a result, implant orientation analysis engine 112 can detect patient motion during the surgical procedure which can affect implant orientation or the accuracy of implant placement. The sensors can also be similarly used intraoperatively to detect the range of motion or position at which the implant dislocates. In addition, the intraoperative motion capture data can be used to ensure that the orientation of the implant in the subject user 140 matches the desired preoperative implant orientation and execution of the entire surgical plan (i.e., intraoperative verification). When assessing the spine, for example, the 3D motion capture data 144 can be used to identify known anatomical landmarks intraoperatively, and implant orientation analysis engine 112 can verify the spinal alignment or implant orientation achieved during surgery, as compared to the planned alignment or orientation. This process is currently performed using radiographs or CT scans, but the use of 3D motion capture data 144 can reduce or eliminate the use of radiation, provide more accurate measurements, and reduce time in acquisition of the information. When assessing the hip after total hip replacement, for example, the 3D motion capture data 144 can be used to identify known anatomical landmarks intraoperatively, and implant orientation analysis engine 112 can verify the acetabular and femoral implant orientation achieved during surgery, as compared to the planned alignment or orientation. This process is currently performed using radiographs or CT scans, but the use of 3D motion capture data 144 can reduce or eliminate the use of radiation, provide more accurate measurements, and reduce time in acquisition of the information. The motion capture data received prior to or during the surgical procedure can be can also be outputted and provided to any delivery device for implant placement (e.g., robotic, computer-assisted navigation, fluoroscopic guidance, augmented reality, patient-specific guides, 3D printed implants).

Furthermore, the dynamic 3D motion capture system described herein can be used in post-surgical applications. For example, the sensors can capture patient motion data after the implant has been received to compare the range of motion to that prior to the surgery to evaluate the effectiveness of the procedure, monitor the range of motion of the joints or implanted prosthesis, monitor the progress and correctness of physical therapy/rehabilitation exercises, track activity levels and motion, etc. In addition, the sensors can be used, together with a preoperative plan, for a planned revision of a hip replacement. For example, the motion capture sensors 142 can be placed on subject user 140 while they perform certain physical movements. Implant orientation analysis engine 112 can receive the resulting 3D motion capture data 144 and perform a similar analysis. In one embodiment, rather than determining a proposed orientation, implant orientation analysis engine 112 can instead analyze the actual orientation of an already implanted surgical implant to determine the cause of dislocation, etc. When assessing spinal alignment and motion after surgical intervention, implant orientation analysis engine 112 can evaluate changes within the instrumented segments, as well as in segments proximal (cephalad) or distal (caudal) to the instrumented segments.

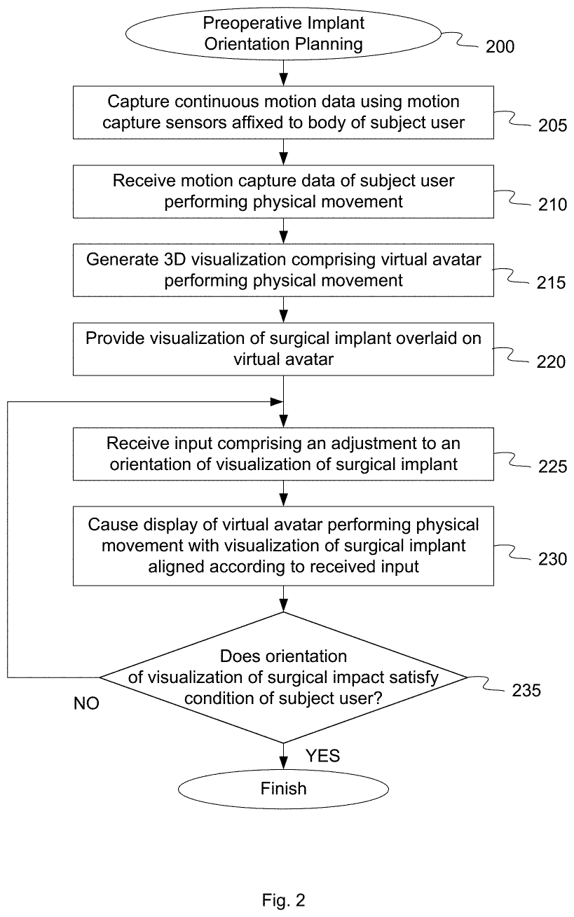

FIG. 2 is a flow diagram illustrating method of preoperative implant orientation planning in accordance with one or more aspects of the present disclosure. The method 200 may be performed by processing logic that comprises hardware (e.g., circuitry, dedicated logic, programmable logic, microcode, etc.), software (e.g., instructions run on a processor to perform hardware simulation), firmware, or a combination thereof In one embodiment, method 200 may be performed by computing device 110 including implant orientation analysis engine 112, as shown in FIG. 1.

Referring to FIG. 2, at block 205, method 200 captures continuous 3D motion capture data 144 corresponding to a subject user 140 using a plurality of motion capture sensors 142 affixed to one or more body parts of the subject user 140 while the subject user 140 is performing a physical movement. In one embodiment, the motion capture sensors 142 are wireless inertial sensors, each including a gyroscope, magnetometer, accelerometer, and/or other components to measure relative positional data, rotational data, acceleration data, and/or other data. The 3D motion capture data 144 includes data representing dynamic motion of at least one of a bone or a joint of the subject user 140 associated with performing the physical movement.

In one embodiment, the motion capture sensors are 142 calibrated to the body of the subject user 140 while the subject user 140 establishes a pose, providing a baseline orientation of the sensors on the respective body parts in a known orientation across the three axes. Computing device 110 sends a signal to the sensors to begin the recording. The subject user 140 performs the protocol movements that will be measured providing movement data such as range of motion, symmetry of joint movement, stability performance, etc. The sensors then send the data back the computing device 110.

At block 210, method 200 receives the 3D motion capture data 144 corresponding to a subject user 140 performing the physical movement. In one embodiment, computing device 110 receives the 3D motion capture data 144 from the motion capture sensors 142 over a wireless communication link (e.g., Bluetooth). In other embodiments, the 3D motion capture data 144 may have been previously captured and stored in a database or other data store, such as repository 120. In one embodiment, the 3D motion capture data 144 is accompanied by a request or instruction to perform a surgical implant orientation analysis to generate a preoperative surgical plan for the subject user 140 pertaining to an orientation of the surgical implant that will be optimized for the physical movement. The request may be received from a user of computing device 110, from a user of a client device coupled to computing device 110 via network 130, or from some other requestor. In one embodiment, implant orientation analysis engine 112 receives the 3D motion capture data 144 and stores the 3D motion capture data 144 in repository 120.

In one embodiment, implant orientation analysis engine 112 can run algorithms that take the raw sensor data and compute human readable motion analysis, for example, taking quaternion sensor data and computing Euler angles relative to the three axis of rotation of bone segments. This can then be converted into joint movement data such as internal/external rotation, abduction/adduction and flexion/extension of a joint (e.g., hip, knee, shoulder, etc.), or bony segment (e.g., femur, tibia/fibula, humerus, radius ulna, vertebra, etc.), as well as joint and skeletal contact stresses and joint reaction forces. Furthermore, the bone and joint movement data can take x-ray angle data to make an off-set adjustment of the initial calibration data. For example, the initial calibration may assume the spine has zero forward or backward bend. The x-ray data may provide initial forward and backward angle bends for the segments of the spine, this can be an input parameter to provide the initial orientation of the segments of the spine from which the relative movement data of the sensors can be offset. The algorithm will compute the segment and joint movements frame by frame captured by the sensors and map out the data in graph form. This information is readily accessible on the computing device right after the capture without human intervention or adjustment. The 3D motion capture sensors can capture continuous movement data across multiple planes, which offers a substantial improvement over static 2D images.

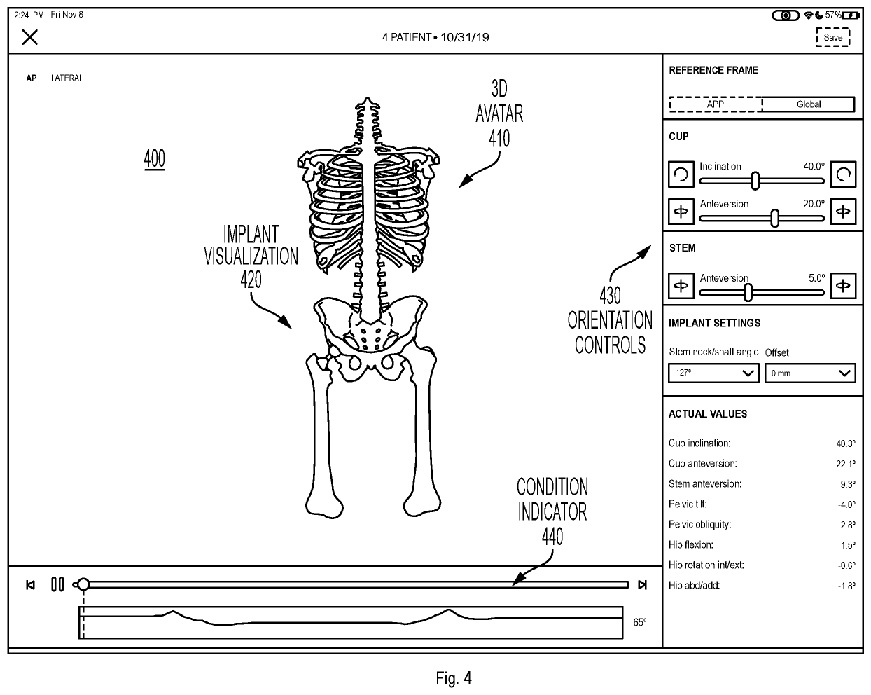

At block 215, method 200 generates, based on the 3D motion capture data 144, a 3D visualization comprising a virtual avatar performing the physical movement. FIG. 4 illustrates one example of a 3D visualization 400 showing a virtual avatar 410 performing the physical movement (in this case moving from a standing position to a seated position). In one embodiment, the 3D visualization 400 is based on 3D motion capture data 144 corresponding to subject user 140 performing the physical movement. The 3D motion capture data 144 can include one or more of positional data, rotational data, or acceleration data measured by a plurality of motion capture sensors 142. In one embodiment, the 3D avatar 410 is represented as a skeleton including at least a portion of the skeletal structure of the subject user 140. The portion of the skeletal structure can include the bones and/or joints of the subject user 140 associated with where the surgical implant will be positioned during the surgical procedure.

At block 220, method 200 provides a visualization of a surgical implant overlaid on the virtual avatar. In the example illustrated in FIG. 4, the surgical implant is a replacement hip joint to be inserted between the pelvis and the femur of the subject user 140. Accordingly, the visualization of the surgical implant 420 can be overlaid on, or otherwise displayed in conjunction with, virtual avatar 410. In other embodiments, the surgical implant can be any other type of implant designed for insertion to some other part of the body (e.g., shoulder, knee, spine, etc.).

At block 225, method 200 receives input comprising an adjustment to an orientation of the visualization of the surgical implant 420 relative to the at least one of the bone or the joint. Implant orientation analysis engine 112 can receive input, such as user input data 146, from a surgeon, or other health professional, including an orientation of the visualization of the surgical implant 420 relative to at least one of a bone or a joint of the virtual avatar 410. In one embodiment, the input can include a value corresponding to at least one of six degrees of freedom for a component of the surgical implant. The six degrees of freedom can include thee three rotational values (i.e., angles) and three translational values (e.g., length, offset, width, height) each defined according to a known plane. In addition to the orientation of the surgical implant, this input also accounts for a resultant skeletal orientation (e.g., leg length, offset in hips, alignment with long bone or spinal osteotomy). In one embodiment, the user interface includes a number of orientation controls 430 through which the user can provide input data 146 to adjust the orientation of the visualization of the surgical implant 420. For example, the orientation controls 430 can include a number of sliders which can be used to adjust the orientation of one or more components of the visualization of the surgical implant 420. In the example illustrated in FIG. 4, the visualization 420 of the surgical implant includes an acetabular cup component affixed to the pelvis, and a stem component affixed to the femur. The stem component is attached to a ball which rests within the cup component, effectively attaching the stem component to the cup component. In this embodiment, the orientation controls 430 allow for adjustment of the orientation of the cup component and the stem component separately. For example, the orientation controls 430 include a slider corresponding to an angle of inclination of the cup component, an angle of anteversion of the cup component, and an angle of anteversion of the stem component. The angles of inclination and anteversion can be defined according to various different references frames which can also be specified in orientation controls 430. For example, the various different reference frames can include a global reference frame (e.g., relative to the floor or to a gravity plumb line) or an anterior pelvic plane (APP) reference frame (e.g., relative to a plane defined by the two anterior superior iliac spines and the pubic symphysis). In other embodiments, the orientation controls 430 can include other controls, such as controls for a size and position of the surgical implant (e.g., the length of the stem component, length of the implanted femoral head), an offset of the surgical implant, or other inputs.

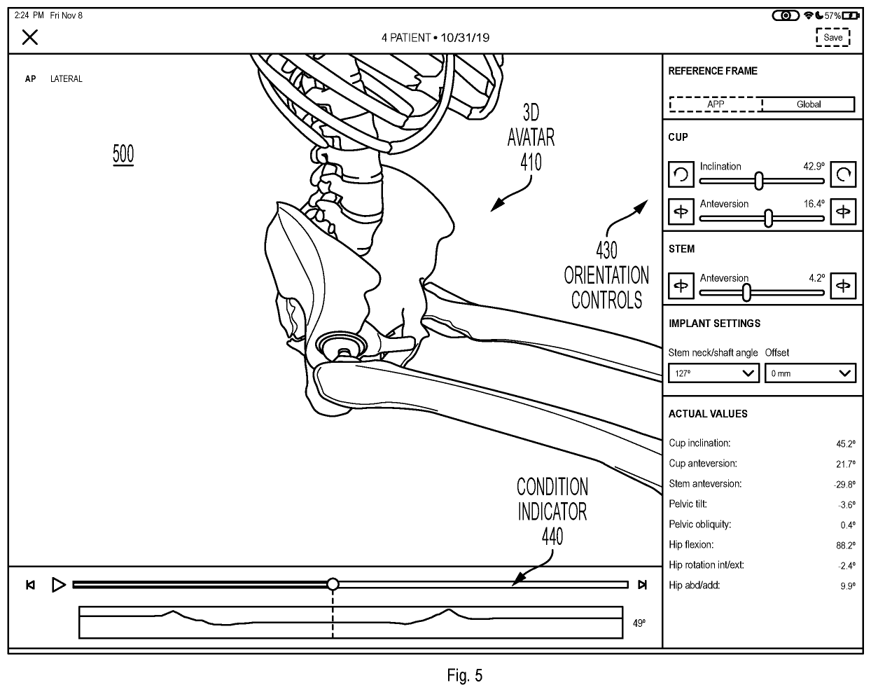

At block 230, method 200 causes display of virtual avatar 410 performing the physical movement with the visualization of the surgical implant 420 aligned according to the input received via orientation controls 430. Given that the 3D motion capture data 144 is continuous for the entire physical movement, changes in the positioning of the bones and joints of the subject user 140 as they perform the physical movement are reflected in the virtual avatar 410. For example, the 3D visualization 400 of FIG. 4 shows the virtual avatar in a standing position seen from the front, while the 3D visualization 500 of FIG. 5 shows the virtual avatar 410 in a seated position. Note that the point of view in FIG. 5 has also been rotated obliquely to the side and zoomed in, as is permitted by the 3D visualization. Although not illustrated in the Figures, the 3D visualization effectively includes a video, based on the 3D motion capture data 144, which can show the positioning of the bones and joints of the subject user 140 at virtually any point in time as they are performing the physical movement.

At block 235, method 200 determines whether the orientation of the visualization of the surgical implant satisfies a condition pertaining to the subject user performing the physical movement. The condition can include, for example, whether the visualization of the surgical implant 420 will come within a threshold margin of an angle of impingement while the virtual avatar 410 is performing the physical movement. Other conditions that can be evaluated include whether a given orientation improves or maximizes the contact area between the surgical implant and the bone in order to reduce edge loading and implant wear, whether a given orientation is optimized for stress distribution to prevent wear, loosening, and/or breakage of the surgical implant, whether the center of pressure of a ball component within a cup component of the surgical implant moves too near the edge of the cup component in order to prevent dislocation, whether contact stresses reach a predetermined threshold, etc. In one embodiment, implant orientation analysis engine 112 monitors various factors associated with the visualization of the surgical implant 420 (e.g., positioning, forces, stress, pressure, etc.) for a given orientation of the visualization of the surgical implant 420 during the physical movement.

In one embodiment, the user interface includes a condition indicator 440 that indicates whether the factors associated with the visualization of the surgical implant 420 satisfy the condition for a given orientation of the visualization of the surgical implant 420 during the physical movement. For example, the condition indicator 440 can include a timeline showing a calculated value relative to the condition. In the example illustrated in FIG. 4, the condition is related to whether the visualization of the surgical implant 420 will come within a threshold margin of an angle of impingement while the virtual avatar 410 is performing the physical movement. The angle of impingement can be reached when a bone of the 3D avatar 410 contacts either another bone or a component of the visualization of the surgical implant 420, or when two components of the visualization of the surgical implant 420 contact each other (e.g., when the stem component contacts the cup component). Impingement is generally sought to be avoided as it can be painful for the subject user 140 and can result in dislocation of the surgical implant. As illustrated, the angle of impingement is reached when the graph reaches the bottom (i.e., x axis of the timeline). Thus, the graph shows how close the visualization of the surgical implant 420 comes to the angle of impingement over a period of time during which the physical movement is performed. In one embodiment, implant orientation analysis engine 112 defines a threshold margin of the angle of impingement (e.g., within 10%) and can generate a warning when that threshold margin is reached. For example, the graph can change color (e.g., turn red) when the threshold margin is reached.

In one embodiment, the condition indicator 440 is updated in response to changes to the orientation of the visualization of the surgical implant 420 made via the orientation controls 430. For example, in response to a change in the orientation, implant orientation analysis engine 112 can recalculate the factors that contribute to whether the condition is satisfied at any point (or at every point) during the performing of the physical movement. In one embodiment, implant orientation analysis engine 112 determines that the condition is satisfied if the visualization of the surgical implant 420 does not reach the angle of impingement during the performing of the physical movement. In another embodiment, implant orientation analysis engine 112 determines that the condition is satisfied if the visualization of the surgical implant 420 does not reach the threshold margin of the angle of impingement during the performing of the physical movement. When other conditions are used, implant orientation analysis engine 112 can consider other criteria to determine whether the condition is satisfied.

In one embodiment, if implant orientation analysis engine 112 determines that the condition is satisfied (e.g., that there will be no impingement), method 200 can end as a valid surgical plan has been developed. If, however, implant orientation analysis engine 112 determines that the condition is not satisfied (e.g., that there will be impingement at some point), method 200 can return to block 225 and receive additional input (e.g., user input data 146 or from an internal algorithm designed to make optimizations) comprising an adjustment to the orientation of the visualization of the surgical implant 420. Method 200 can continue through blocks 225, 230 and 235 until the condition is satisfied.

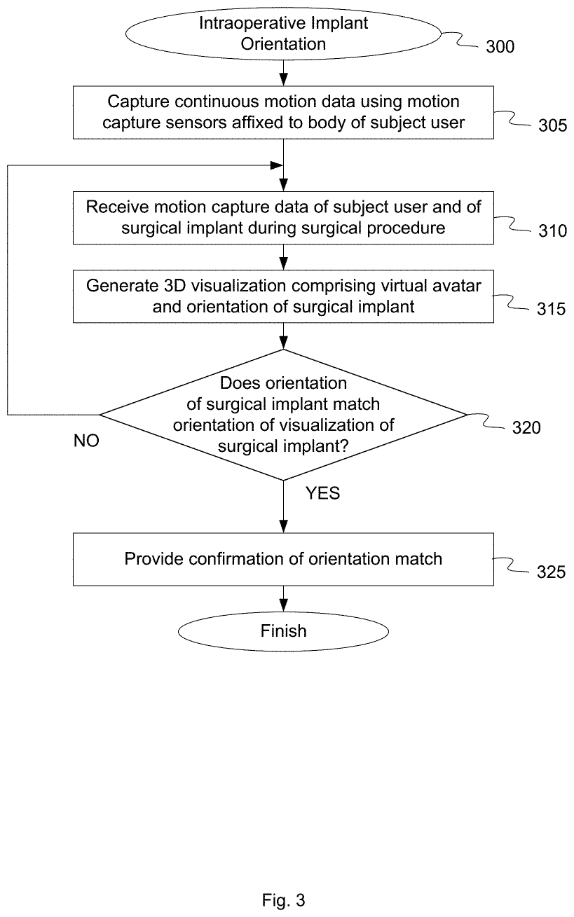

FIG. 3 is a flow diagram illustrating method of intraoperative implant orientation in accordance with one or more aspects of the present disclosure. The method 300 may be performed by processing logic that comprises hardware (e.g., circuitry, dedicated logic, programmable logic, microcode, etc.), software (e.g., instructions run on a processor to perform hardware simulation), firmware, or a combination thereof In one embodiment, method 300 may be performed by computing device 110 including implant orientation analysis engine 112, as shown in FIG. 1.

Referring to FIG. 3, at block 305, method 300 captures continuous 3D motion capture data 144 corresponding to a subject user 140 using a plurality of motion capture sensors 142 affixed to one or more body parts of the subject user 140 and optionally to a surgical implant, while the subject user 140 is undergoing a surgical procedure to receive the surgical implant. In one embodiment, the 3D motion capture data 144 includes data representing a location of at least one of a bone or a joint of the subject user 140 and a relative location and orientation of the surgical implant. For example, the 3D motion capture data 144 can identify any movements of the subject user 140 during the surgical procedure and precisely identify the orientation of the surgical implant relative to the at least one of the bone or the joint of the subject user 140.

At block 310, method 300 receives the 3D motion capture data 144 corresponding to the subject user 140 and the surgical implant. In one embodiment, computing device 110 receives the 3D motion capture data 144 from the motion capture sensors 142 over a wireless communication link (e.g., Bluetooth). In one embodiment, the 3D motion capture data 144 is accompanied by a request or instruction to perform a surgical implant orientation analysis pertaining to an orientation of the surgical implant relative to the at least one of the bone or the joint of the subject user 140. The request may be received from a user of computing device 110, from a user of a client device coupled to computing device 110 via network 130, or from some other requestor. In one embodiment, implant orientation analysis engine 112 receives the 3D motion capture data 144 and stores the 3D motion capture data 144 in repository 120.

At block 315, method 300 generates, based on the 3D motion capture data 144, a 3D visualization comprising a virtual avatar and showing the relative position and orientation of the surgical implant. As described above, the 3D avatar can be represented as a skeleton including at least a portion of the skeletal structure of the subject user 140. The portion of the skeletal structure can include the bones and/or joints of the subject user 140 associated with where the surgical implant is positioned during the surgical procedure.

At block 320, method 300 determine whether the orientation of the surgical implant during the surgical procedure matches the orientation of the visualization of the surgical implant that satisfied the condition pertaining to the subject user performing the physical movement. In one embodiment, implant orientation analysis engine 112 compares the orientation of the surgical implant during the surgical procedure (e.g., at least one of an angle of inclination or an angle of anteversion) to the orientation that was determined to satisfy the condition during the preoperative planning performed in method 200. In one embodiment, implant orientation analysis engine 112 maintains certain threshold tolerances (e.g., +/−3 degrees), which when satisfied, the orientations can be considered to match. If implant orientation analysis engine 112 determines that the orientations do not match, method 300 can return to block 310 and receive updated motion capture data in response to a manual adjustment of the orientation of the surgical implant made by the surgeon or other health professional. Method 300 can continue through blocks 310, 315, and 320 until the orientations match. Once implant orientation analysis engine 112 determines that the orientations match, at block 325, method 300 provides a confirmation of the orientation match.

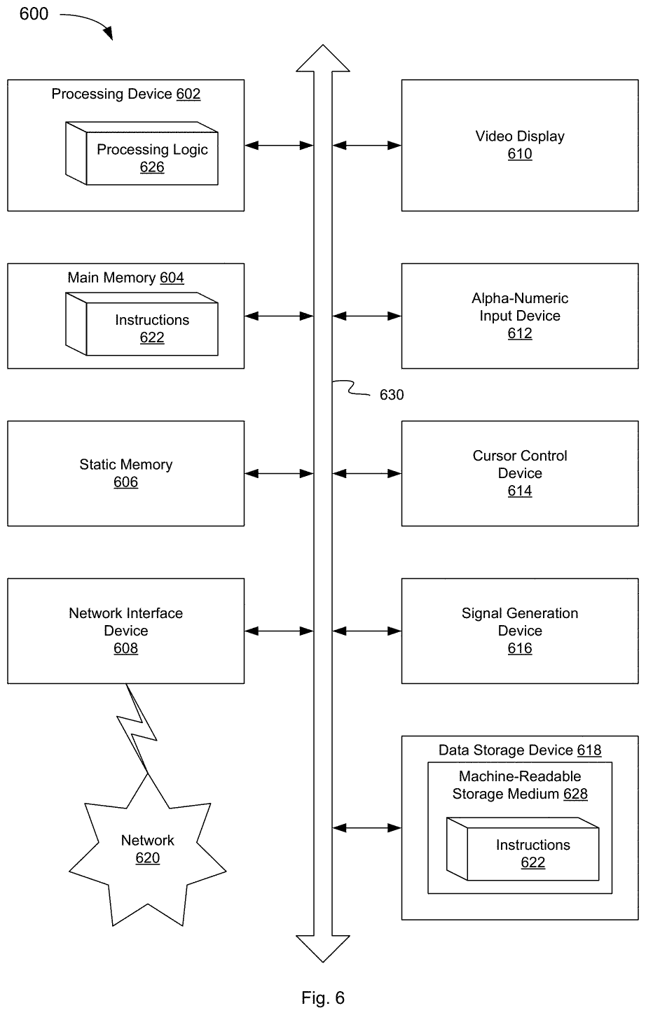

FIG. 6 depicts an example computer system 600 which can perform any one or more of the methods described herein, in accordance with one or more aspects of the present disclosure. In one example, computer system 600 may correspond to a computing device, such as computing device 110, capable of executing implant orientation analysis engine 112 of FIG. 1. The computer system 600 may be connected (e.g., networked) to other computer systems in a LAN, an intranet, an extranet, or the Internet. The computer system 600 may operate in the capacity of a server in a client-server network environment. The computer system 600 may be a personal computer (PC), a tablet computer, a set-top box (STB), a personal Digital Assistant (PDA), a mobile phone, a camera, a video camera, or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The exemplary computer system 600 includes a processing device 602, a main memory 604 (e.g., read-only memory (ROM), flash memory, dynamic random access memory (DRAM) such as synchronous DRAM (SDRAM)), a static memory 606 (e.g., flash memory, static random access memory (SRAM)), and a data storage device 618, which communicate with each other via a bus 630.

Processing device 602 represents one or more general-purpose processing devices such as a microprocessor, central processing unit, or the like. More particularly, the processing device 602 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 602 may also be one or more special-purpose processing devices such as an application specific integrated circuit (ASIC), a field programmable gate array (FPGA), a digital signal processor (DSP), network processor, or the like. The processing device 602 is configured to execute instructions for performing the operations and steps discussed herein.

The computer system 600 may further include a network interface device 608. The computer system 600 also may include a video display unit 610 (e.g., a liquid crystal display (LCD) or a cathode ray tube (CRT)), an alphanumeric input device 612 (e.g., a keyboard), a cursor control device 614 (e.g., a mouse), and a signal generation device 616 (e.g., a speaker). In one illustrative example, the video display unit 610, the alphanumeric input device 612, and the cursor control device 614 may be combined into a single component or device (e.g., an LCD touch screen).

The data storage device 618 may include a computer-readable medium 628 on which the instructions 622 (e.g., implementing implant orientation analysis engine 112) embodying any one or more of the methodologies or functions described herein is stored. The instructions 622 may also reside, completely or at least partially, within the main memory 604 and/or within the processing device 602 during execution thereof by the computer system 600, the main memory 604 and the processing device 602 also constituting computer-readable media. The instructions 622 may further be transmitted or received over a network via the network interface device 608.

While the computer-readable storage medium 628 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

Although the operations of the methods herein are shown and described in a particular order, the order of the operations of each method may be altered so that certain operations may be performed in an inverse order or so that certain operation may be performed, at least in part, concurrently with other operations. In certain implementations, instructions or sub-operations of distinct operations may be in an intermittent and/or alternating manner.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

In the above description, numerous details are set forth. It will be apparent, however, to one skilled in the art, that the aspects of the present disclosure may be practiced without these specific details. In some instances, well-known structures and devices are shown in block diagram form, rather than in detail, in order to avoid obscuring the present disclosure.

Some portions of the detailed descriptions above are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving,” “determining,” “selecting,” “storing,” “setting,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present disclosure also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear as set forth in the description. In addition, aspects of the present disclosure are not described with reference to any particular programming language. It will be appreciated that a variety of programming languages may be used to implement the teachings of the present disclosure as described herein.

Aspects of the present disclosure may be provided as a computer program product, or software, that may include a machine-readable medium having stored thereon instructions, which may be used to program a computer system (or other electronic devices) to perform a process according to the present disclosure. A machine-readable medium includes any procedure for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.).

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

1. A method comprising:
receiving first continuous motion capture data representing dynamic motion of at least one of a bone or a joint of the subject user while performing a physical movement;
generating, based on the continuous motion capture data, a three dimensional (3D) visualization comprising a virtual avatar performing the physical movement;
providing a visualization of a surgical implant overlaid on the virtual avatar;
receiving input comprising an adjustment to an orientation of the visualization of the surgical implant relative to the at least one of the bone or the joint; and
determining whether the orientation of the visualization of the surgical implant satisfies a condition pertaining to the subject user performing the physical movement.

2. The method of claim 1, wherein receiving the input comprises receiving values corresponding to all six degrees of freedom for each individual component of the surgical implant.

3. The method of claim 2, wherein the values corresponding to all six degrees of freedom comprise rotational values and translational values defined according to a known plane.

4. The method of claim 1, wherein determining whether the orientation of the visualization of the surgical implant satisfies the condition pertaining to the subject user comprises determining whether the visualization of the surgical implant will come within a threshold margin of an angle of impingement while the virtual avatar is performing the physical movement.

5. The method of claim 1, further comprising:
capturing the first continuous motion capture data using a plurality of motion capture sensors affixed to one or more body parts of the subject user while the subject user is performing the physical movement.

6. The method of claim 5, wherein the first continuous motion capture data comprises one or more of positional data, rotational data, or acceleration data measured by the plurality of motion capture sensors.

7. The method of claim 1, further comprising:
causing display of the virtual avatar performing the physical movement with the visualization of the surgical implant aligned according to the received input.

8. The method of claim 1, further comprising:
receiving second continuous motion capture data representing dynamic motion of the at least one of the bone or the joint of the subject user and of the surgical implant during a surgical procedure; and
verifying that an orientation of the surgical implant during the surgical procedure matches the orientation of the visualization of the surgical implant that satisfied the condition pertaining to the subject user performing the physical movement.

9. A system comprising:
a memory device storing instructions;
a processing device coupled to the memory device, the processing device to execute the instructions to:
receive first continuous motion capture data representing dynamic motion of at least one of a bone or a joint of the subject user while performing a physical movement;
generate, based on the continuous motion capture data, a three dimensional (3D) visualization comprising a virtual avatar performing the physical movement;
providing a visualization of a surgical implant overlaid on the virtual avatar;
receive input comprising an adjustment to an orientation of the visualization of the surgical implant relative to the at least one of the bone or the joint; and
determine whether the orientation of the visualization of the surgical implant satisfies a condition pertaining to the subject user performing the physical movement.

10. The system of claim 9, wherein the input comprises values corresponding to all six degrees of freedom for each individual component of the surgical implant.

11. The system of claim 9, wherein the values corresponding to all six degrees of freedom comprise rotational values and translational values defined according to a known plane.

12. The system of claim 9, wherein the processing device further to:
capture the first continuous motion capture data using a plurality of motion capture sensors affixed to one or more body parts of the subject user while the subject user is performing the physical movement, wherein the first continuous motion capture data comprises one or more of positional data, rotational data, or acceleration data measured by the plurality of motion capture sensors.

13. The system of claim 9, wherein the processing device further to:
cause display of the virtual avatar performing the physical movement with the visualization of the surgical implant aligned according to the received input.

14. The system of claim 9, wherein the processing device further to:
receive second continuous motion capture data representing dynamic motion of the at least one of the bone or the joint of the subject user and of the surgical implant during a surgical procedure; and
verify that an orientation of the surgical implant during the surgical procedure matches the orientation of the visualization of the surgical implant that satisfied the condition pertaining to the subject user performing the physical movement.

15. A non-transitory computer-readable storage medium storing instructions that, when executed by a processing device, cause the processing device to:
receive first continuous motion capture data representing dynamic motion of at least one of a bone or a joint of the subject user while performing a physical movement;
generate, based on the continuous motion capture data, a three dimensional (3D) visualization comprising a virtual avatar performing the physical movement;
provide a visualization of a surgical implant overlaid on the virtual avatar;
receive input comprising an adjustment to an orientation of the visualization of the surgical implant relative to the at least one of the bone or the joint; and
determine whether the orientation of the visualization of the surgical implant satisfies a condition pertaining to the subject user performing the physical movement.

16. The non-transitory computer-readable storage medium of claim 15, wherein the input comprises values corresponding to all six degrees of freedom for each individual component of the surgical implant, and wherein the values corresponding to all six degrees of freedom comprise rotational values and translational values defined according to a known plane.

17. The non-transitory computer-readable storage medium of claim 15, wherein to determine whether the orientation of the visualization of the surgical implant satisfies the condition pertaining to the subject user, the processing device to determine whether the visualization of the surgical implant will come within a threshold margin of an angle of impingement while the virtual avatar is performing the physical movement.

18. The non-transitory computer-readable storage medium of claim 15, wherein the processing device further to:
capture the first continuous motion capture data using a plurality of motion capture sensors affixed to one or more body parts of the subject user while the subject user is performing the physical movement, wherein the first continuous motion capture data comprises one or more of positional data, rotational data, or acceleration data measured by the plurality of motion capture sensors.

19. The non-transitory computer-readable storage medium of claim 15, wherein the processing device further to:
cause display of the virtual avatar performing the physical movement with the visualization of the surgical implant aligned according to the received input.

20. The non-transitory computer-readable storage medium of claim 15, wherein the processing device further to:
receive second continuous motion capture data representing dynamic motion of the at least one of the bone or the joint of the subject user and of the surgical implant during a surgical procedure; and
verify that an orientation of the surgical implant during the surgical procedure matches the orientation of the visualization of the surgical implant that satisfied the condition pertaining to the subject user performing the physical movement.
